(19) United States
(12) Patent Application Publication
(10) Pub. No.: US 2024/0189380 A1
(43) Pub. Date: Jun. 13, 2024

(54) COMPOSITIONS FOR INHIBITING EHP INFECTION IN SHRIMP AND RELATED METHODS

(71) Applicant: KEMIN INDUSTRIES, INC., DES MOINES, IA (US)

(72) Inventors: Harikumar SAMPATH, Kanchipuram (IN); Mukkalil RAJALEKSHMI, Chennai (IN)

(73) Assignee: KEMIN INDUSTRIES, INC., DES MOINES, IA (US)

(21) Appl. No.: 18/510,941

(22) Filed: Nov. 16, 2023

(30) Foreign Application Priority Data
Nov. 17, 2022 (IN) 202211065837

(51) Int. Cl. A61K 36/53; A23K 10/30; A23K 50/80; A61K 36/71; A61K 47/44; A61P 31/10

(52) U.S. Cl. CPC A61K 36/53; A23K 10/30; A23K 50/80; A61K 36/71; A61K 47/44; A61P 31/10

(57) ABSTRACT

The present invention relates methods for administering compositions comprising thymoquinone in an amount effective to inactivate the EHP spores and reduce the EHP infection rate, where the thymoquinone may be derived from Monarda didyma, Monarda fistulosa, or Nigella sativa. The present invention further relates to methods for controlling the proliferation of EHP in aquaculture, specifically controlling the spread of EHP infection in shrimp populations. Another aspect of the present invention relates to methods for directly inactivating EHP spores to control the intracellular proliferation of EHP by administering compositions containing thymoquinone, for instance compositions containing N. sativa seed oil to shrimp. Another aspect of the present invention relates to compositions containing N. sativa seed extract alone, or in oil, in at least one embodiment in combination with monarda oil, oregano oil, clove oil and cinnamic aldehyde, to control and inhibit the spread of EHP infection and its associated diseases.

### CROSS-REFERENCE TO RELATED APPLICATIONS

The present application claims the benefit of priority to Indian Provisional Patent Application No. 202211065837, filed Nov. 17, 2023, entitled “COMPOSITIONS FOR INHIBITING EHP INFECTION IN SHRIMP AND RELATED METHODS,” the entire disclosure of which is incorporated by reference herein in its entirety.

### BACKGROUND OF THE INVENTION

Hepatopancreatic microsporidiosis (HPM) is a shrimp microsporidian infection caused by an intracellular microsporidian parasite Enterocytozoon hepatopenaei (EHP). Due to limited methods to detect the infection, as well as the absence of methods to control the spread of EHP, the shrimp-farming industry has suffered major economic losses due to EHP infection. Thawatchai, C., et al. (2021), The shrimp microsporidian Enterocytozoon hepatopenaei (EHP): Biology, pathology, diagnostics and control, Journal of Invertebrate Pathology, 186:107458; Thitamadee S., et al. (2016), Review of current disease threats for cultivated penaeid shrimp in Asia, Aquaculture, 452: 69-87; Chao Ma, et al. (2021), Rapid detection of Enterocytozoon hepatopenaei infection in shrimp with a Real-Time isothermal recombinase polymerase amplification assay, Frontiers in Cellular and Infection Microbiology, Vol. 11, 1-8.

Shrimp hepatopancreas (HP) is a central organ for secretion of digestive enzymes, absorption, and storage of nutrients. EHP is an emerging pathogen that primarily infects tubular epithelial cells of the HP and leads to severe growth retardation and size variability among shrimp. EHP is also associated with other bacterial diseases, where shrimp infected with EHP have been found to be highly susceptible to various Vibrio infections resulting in mass mortality. See Han, J. L., et al. (2019), Molecular detection of Enterocytozoon hepatopenaei and Vibrio parahaemolyticus-associated acute hepatopancreatic necrosis disease in Southeast Asian Penaeus vannamei shrimp imported into Korea, Aquaculture, 517:734812. Currently, EHP is a limiting factor in the sustainability of global shrimp production as the disease remains widespread in shrimp farming countries without an effective solution to control the spread.

### SUMMARY OF THE INVENTION

The present invention relates to compositions for controlling the proliferation of EHP in aquaculture, specifically compositions that are capable of controlling the spread of EHP in shrimp populations. One aspect of the present invention relates to compositions containing N. sativa seed extract/oil alone or in combination with essential oils (monarda oil, oregano oil, clove oil and cinnamic aldehyde) that can control and inhibit the spread of EHP infection and its associated diseases. According to at least one embodiment, the compositions of the present invention are administered to shrimp as a feed additive.

Another aspect of the present invention relates to methods leading to the direct inactivation of EHP spores in order to prevent or reduce the infection in shrimp. Another aspect of the present invention relates to administering compositions, such as animal feed additives that are included in animal feed, in an amount that is effective to control the spread of EHP. Another aspect of the present invention relates to orally administering compositions to shrimp in an amount effective to inhibit EHP proliferation in shrimp populations and administering said compositions to shrimp in an amount effective to control HPM.

The inventors have discovered compositions capable of inactivating the EHP polar tube extrusion mechanism (adhesive mechanism), which is a promising development to control the first step of infection called transferring EHP sporoplasm into the host cell, which is an adhesive mechanism.

Further, the inventors have discovered compositions capable of inactivating the EHP post translational modification enzyme Methionine amino peptidase 2 (MetAP2), which holds promise for controlling the multiplication of EHP at an intracellular level. The inventors have surprisingly found compositions that are effective at inhibiting the proliferation of EHP and also deemed safe for orally administering to shrimp, for instance through supplementation in feed.

### DETAILED DESCRIPTION OF THE FIGURES

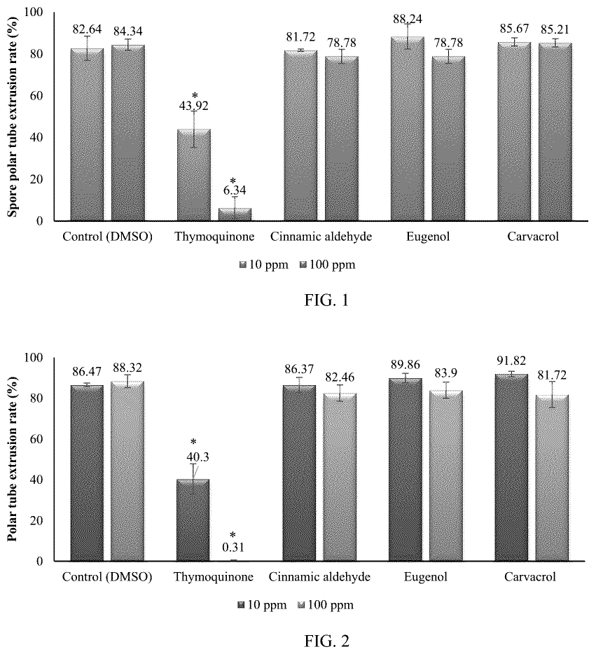

FIG. 1 depicts the effect of bioactive molecules on the polar tube extrusion of the Batch 1 spores. All the data represented as mean±SD, N=3. The asterisk showed the significant difference (p=0.002) in % of extrusion compared to dimethyl sulfoxide (DMSO) control.

FIG. 2 depicts the effect of bioactive molecules on the polar tube extrusion of the Batch 2 spores. All the data represented as mean±SD, N=3. The asterisk showed the significant difference (p=0.002) in % of extrusion compared to DMSO control.

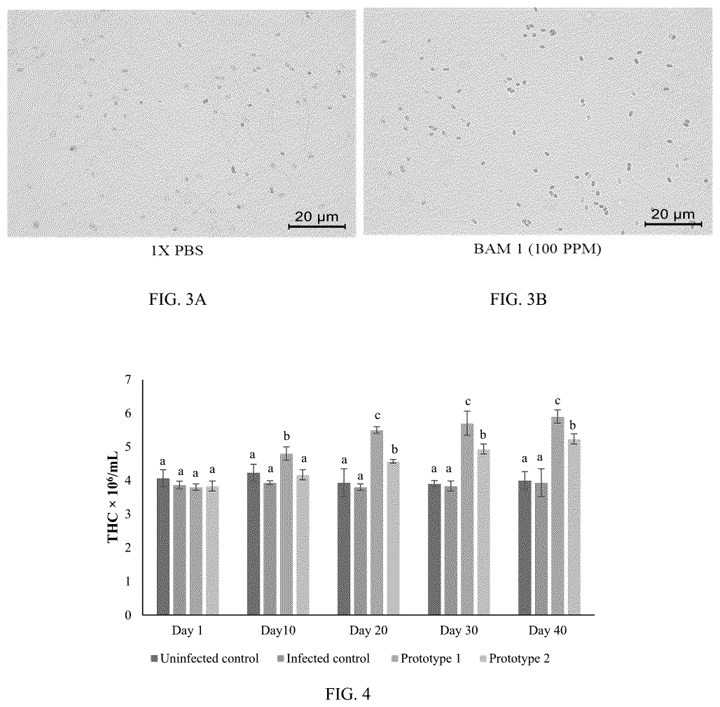

FIGS. 3A and 3B are photomicrographs of polar tube extruded-EHP spores under microscopic examination (100×). Representative images of spores from Batch 2 treated with 100 ppm of thymoquinone (BAM1) and control.

FIG. 4 depicts total hemocyte count at different intervals. Data represented as mean±SD: N=3 (p<0.05).

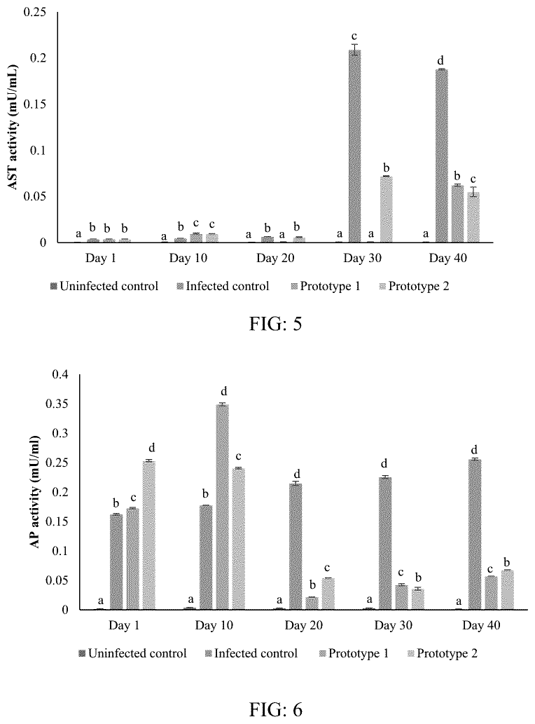

FIG. 5 depicts Aspartate aminotransferase (AST) activity at different intervals. Data represented as mean±SD: N=3 (p<0.05).

FIG. 6 depicts Alkaline phosphatase (AP) activity at different intervals. Data represented as mean±SD: N=3 (p<0.05).

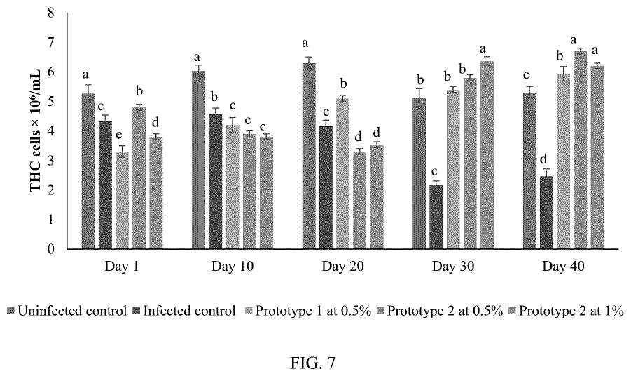

FIG. 7 depicts total hemocyte count at different intervals. Data represented as mean±SD: N=3 (p<0.05).

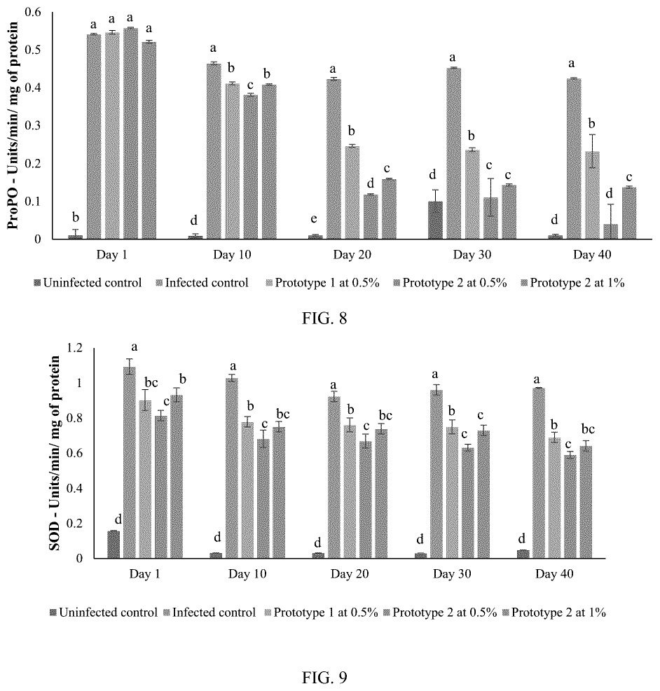

FIG. 8 depicts prophenoloxidase (ProPO) activity at different intervals. Data represented as mean±SD: N=3 (p<0.05).

FIG. 9 depicts superoxide dismutase (SOD) activity at different intervals. Data represented as mean±SD: N=3 (p<0.05).

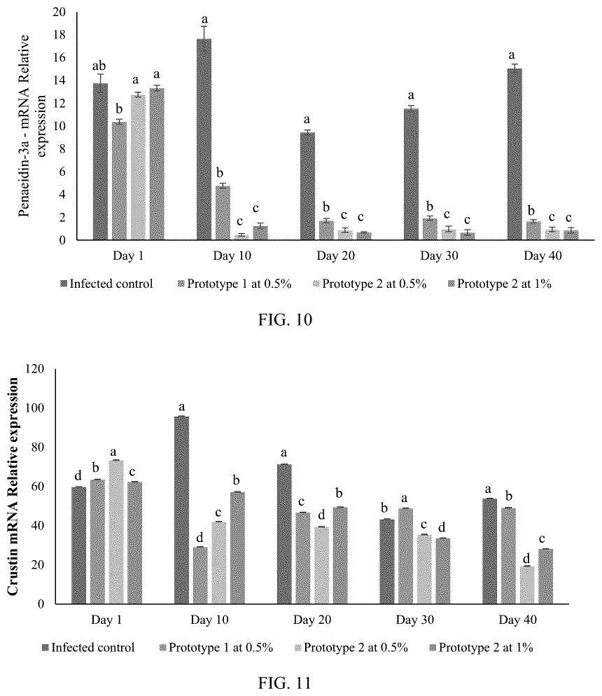

FIG. 10 depicts relative penaeidin-3a gene expression at different intervals. Data represented as mean±SD: N=3 (p<0.05).

FIG. 11 depicts relative crustin gene expression at different intervals. Data represented as mean±SD: N=3 (p<0.05).

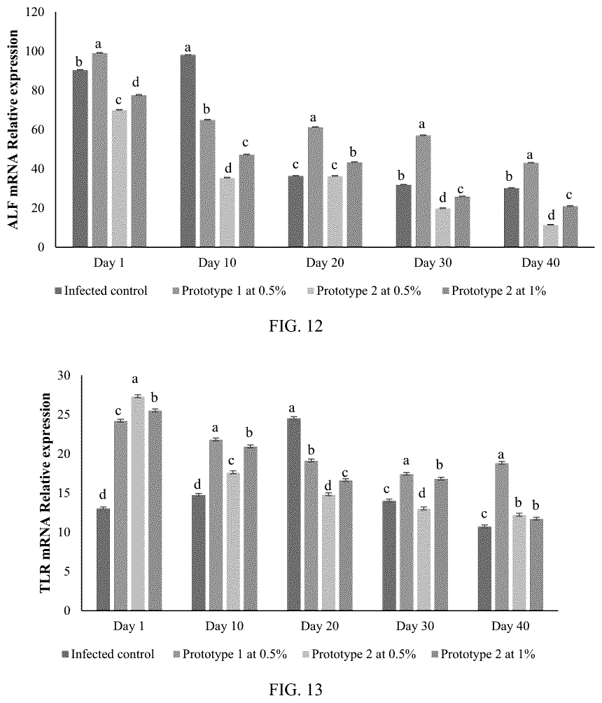

FIG. 12 depicts relative anti-lipopolysaccharide (ALF) factor gene expression at different intervals. Data represented as mean±SD: N=3 (p<0.05).

FIG. 13 depicts relative toll like receptor (TLR) gene expression at different intervals. Data represented as mean±SD: N=3 (p<0.05).

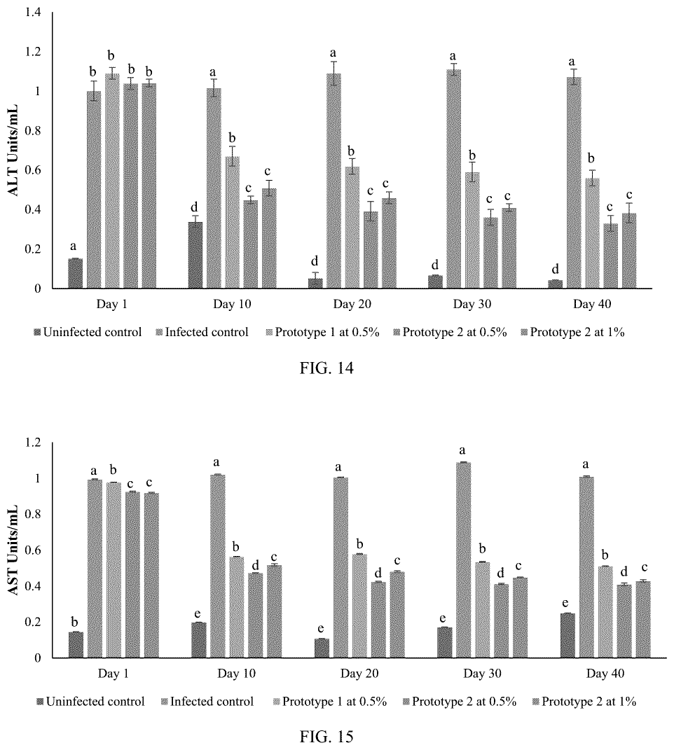

FIG. 14 depicts alanine aminotransferase (ALT) activity at different intervals. Data represented as mean±SD: N=3 (p<0.05).

FIG. 15 depicts aspartate aminotransferase (AST) activity at different intervals. Data represented as mean±SD: N=3 (p<0.05).

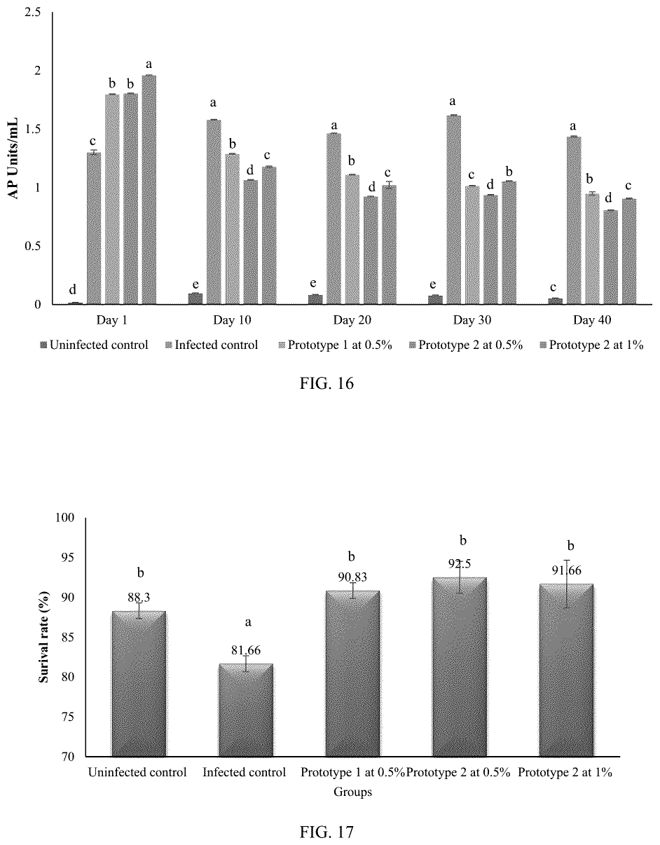

FIG. 16 depicts alkaline phosphatase (AP) activity at different intervals. Data represented as mean±SD: N=3 (p<0.05).

FIG. 17 depicts the survival rate (%) after the bacterial challenge of shrimp using V. parahaemolyticus for 120 h at 2.6×10³ CFU/ml. All values are represented as mean±SD where n=4 (30 shrimp in each replicate) (P<0.05).

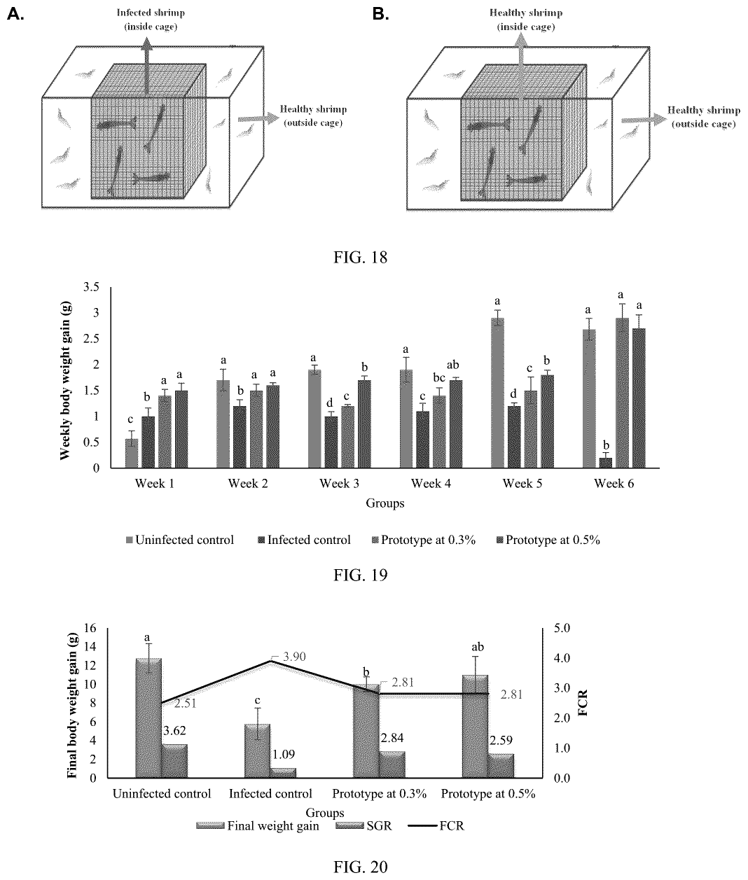

FIG. 18 depicts the cohabitation challenge model (Salachan et. al., 2016). A—model for infected control and treatments groups. B—model for uninfected control

FIG. 19 depicts the weekly body weight gain at different intervals. All the data represented as mean±SD, n=20 (5 shrimp from each replicate) (p<0.05).

FIG. 20 depicts the final weight gain, feed conversion ratio and specific growth rate of different experimental groups. Data represented as mean±SD; n=4 (25 shrimp in each replicate): (P<0.05).

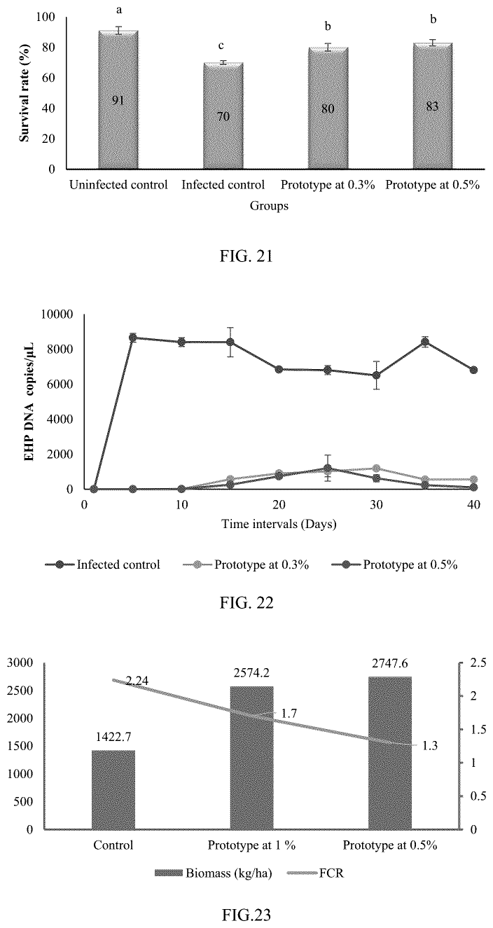

FIG. 21 depicts the Final survival rate (%) of different experimental groups. Data represented as mean±SD; n=4 (25 shrimp in each replicate): (P<0.05).

FIG. 22 depicts the quantification of EHP by qPCR. All values are represented as mean±SD where n=4 (P<0.05)

FIG. 23 depicts the final biomass yield and feed conversion ratio of experimental ponds.

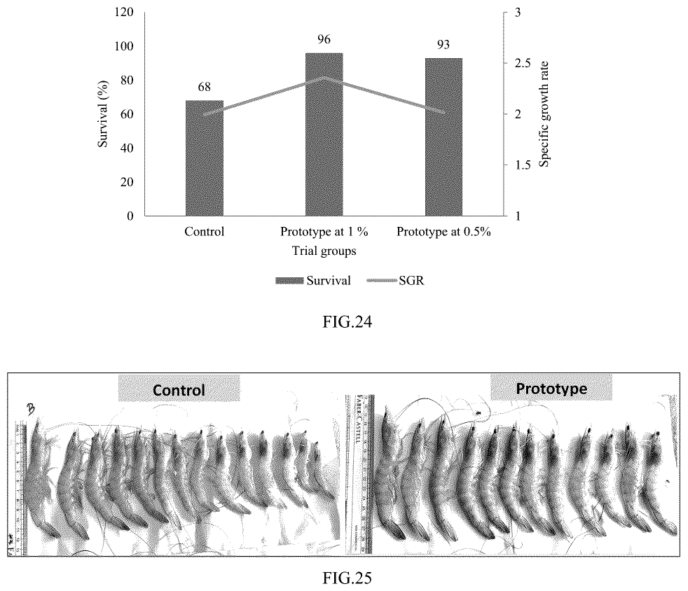

FIG. 24 depicts the final survival and specific growth rate of experimental ponds.

FIG. 25 depicts the final size variations between control and treatment ponds.

### DETAILED DESCRIPTION OF THE INVENTION

Plant derived components are potential sustainable source of antimicrobial chemotypes and remain untapped as potential solutions for controlling pathogens. The inventors have surprisingly discovered that dietary supplementation of Nigella sativa extract/oil alone, or in combination with essential oils, such as monarda oil, oregano oil, clove oil and cinnamon bark oil, demonstrated a statistically significant reduction of EHP infection in shrimp. The inventors also demonstrated that Aquavibra™ (Kemin Industries, Inc.), a combination of cinnamic aldehyde and oregano oil, was capable of achieving considerable reduction of EHP infection. Nigella sativa is an annual herbaceous plant belonging to the Ranunculaceae family. N. sativa seed is rich in terpenes compounds including thymoquinone, p-cymene, γ-terpinene, β-pinene, carvacrol, terpinen-4-ol and longifolene.

The present invention relates to compositions containing N. sativa seed oil for controlling the proliferation of EHP in aquaculture, specifically in amounts capable of controlling the spread of EHP infection in shrimp populations. According to at least one embodiment of the present invention, the composition further comprises at least one essential oil including but not limited to those selected from Nigella sativa seed oil/extract, cinnamic aldehyde, oregano oil, cinnamon bark oil, clove oil, monarda oil or combinations thereof. In a preferred embodiment, the composition contains N. sativa seed oil in combination with oregano oil, monarda oil, clove oil and cinnamic aldehyde. In alternative embodiments, the composition of the present invention may include thymoquinone, thymohydroquinone, dithymoquinone, p-cymene, carvacrol, terpineol, thymol, and combinations thereof.

In certain embodiments, the compositions of the present invention contain N. sativa seed oil in an amount ranging from about 0.1 to 100 percent by weight, for instance 1 to 95 percent by weight, or alternatively 5 to 95 percent by weight. In certain embodiments, the composition further contains oregano oil in an amount ranging from about 0 to 99.9 percent by weight, monarda oil in an amount ranging from about 0 to 99.9 percent by weight, clove oil in and amount ranging from in an amount ranging from about 0 to about 50 percent by weight and cinnamic aldehyde in an amount ranging from about 0 to about 50 percent by weight. In certain embodiments, the composition optionally includes thymoquinone, thymohydroquinone, dithymoquinone, p-cymene, carvacrol, terpineols, and/or thymol, wherein such optional ingredients are present in an amount ranging from about 0.1 to about 50 percent by weight.

Another aspect of the present invention relates to the direct inactivation of EHP spores in order to control the adhesive mechanism and intracellular proliferation by altering post-translational modifications. In some embodiments, orally administering a composition containing an effective amount of N. sativa seed oil results in a reduction of EHP infection by an amount ranging from about 0 to 100 percent, for instance, a 5, 10, 20, 30, 40, 50, 60, 70, 80, 90 or 100 percent reduction of infection.

Another aspect of the present invention relates to compositions containing an effective amount of N. sativa seed oil that result in a reduction of EHP infection, such as a supplement or additive to animal feed, where the composition is included in animal feed in an amount that is effective to control the spread of EHP. According to at least one embodiment, the compositions of the present invention are included in feed at an amount ranging from about 0.1 to 20 g/kg of feed, for instance from about 0.5 to 10 g/kg of feed.

Another aspect of the present invention relates to administering compositions to shrimp in an amount effective to inhibit EHP in shrimp populations and administering compositions to shrimp in an amount effective to control HPM.

Another aspect of the present invention relates to compositions that are capable of inactivating the EHP infection, which involves extrusion of polar tube (an adhesive mechanism). For instance, in some embodiments the EHP infection rate in the shrimp population decreases while EHP spores continue to proliferate.

Another aspect of the present invention relates to compositions that are effective at inhibiting the proliferation of EHP and at the same time safe for administering to shrimp, for instance through supplementation in animal feed. In alternative embodiments, the compositions can be added to the water source. In yet alternative embodiments, the compositions could be applied to water or soil sediments that contain EHP spores.

Another aspect of the present invention relates to administering compositions in an amount effective to inactivate the EHP spores present in the shrimp culture water and shrimp pond soil sediments.

The following examples illustrate the present invention and are not intended to be limiting.

### EXAMPLES

### Example 1

In-silico molecular docking and molecular dynamics (MD) simulations of selected bioactive compounds (carvacrol, α-terpineol, thymoquinone, cinnamic aldehyde and eugenol) and positive controls (Fumagillin and Albendazole) were performed with microsporidian drug target, methionine aminopeptidase 2 (MetAP2), as summarized in Table 1. Results revealed promising inhibitory potential of three lead compounds, carvacrol, α-terpineol and thymoquinone against MetAP2, with binding energies comparable to controls. Molecular mechanics with generalized Born and surface area solvation (MM/GBSA) and Poisson-Boltzmann surface area (MM/PBSA) calculation revealed thermodynamically stable binding energies of the compounds with MetAP2. The binding and inhibition of MetAP2 by the compounds were proved to be thermodynamically favorable as summarized in Table 2 and Table 3.

TABLE 1
Binding affinity of bioactive molecules and positive controls, with amino acids.

| | | Binding | Amino acids & bonds | | | |
|---|---|---|---|---|---|---|
| | | affinity | | | Van | |
| | | (kCal/ | | | der | |
| Lead-CID | Structure | mol) | H Bond | π bond | waals | Alkyl |
| Carvacrol_Structure 2D_CID_10364 | 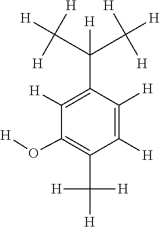 [SMILES: Cc1ccc(C(C)C)cc1O] | −7 | Asp130 | Phe97 | Asp141 His210 Gln337 Glu339 Fe452 | Ile217 |
| Fumagillin_Structure 2D_CID_6917655 | 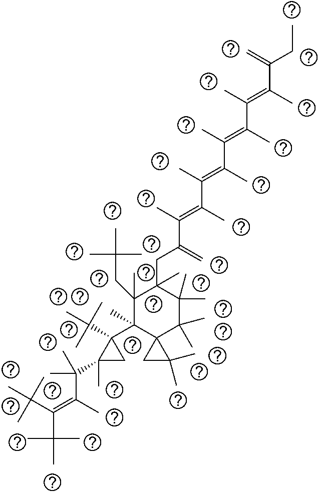 [SMILES: C=C(CC)/C(C)=C(C)/C(C)=C(C)/C(C)=C(C)/C(C)=C(\C)C(=C)CC1(C)C(C)(C)C(C)(C)C2(CC2(C)C)[C@@](C)([C@@]2(C(C)(C)C)C[C@]2(C)C(C)(C)C(C)=C(C(C)(C)C)C(C)(C)C)C1(C)CC(C)(C)C] | −6.8 | Asp130 Thr290 (Carbon Hydrogen Bond) | | Asp141 His210 Fe452 | Phe97 His109 Ile217 Val263 Tyr267 Pro292 Tyr324 |
| Alpha Terpineol_ Structure 2D_CID_17100 | 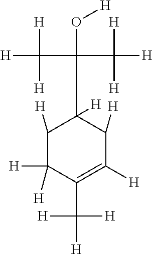 [SMILES: CC1=CCC(C(C)(C)O)CC1] | −6.5 | | (Pi sigma) Phe97 Tyr324 | Ile217 His261 Phe262 | His109 Val263 Pro292 |
| Eugenol_Structure 2D_CID_3314 | 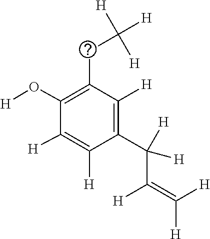 [SMILES: C=CCc1ccc(O)c(CC)c1] | −6.4 | His210 Glu243 | Phe97 His109 | Fe451 | Ile217 His218 His261 Tyr324 |
| Cinnamic aldehyde_Structure 2D_CID_637511 | 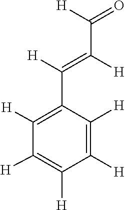 | −6.4 | | His109 | Phe97 His210 | Ile217 Pro292 |
| Albendazole_ Structure 2D_CID_2082 | 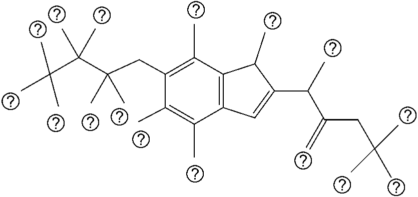 [SMILES: C=C(CC(C)(C)C)C(C)C1=Cc2c(C)c(C)c(CC(C)(C)C(C)(C)C(C)(C)C)c(C)c2C1C] | −6.4 | His208 Glu243 | His109 His210 (Pi Anion) Fe451 (Pi sulfur) Phe97 | | Ile217 His261 Pro292 Tyr324 |
| Thymoquinone_ Structure 2D_CID_10281 | 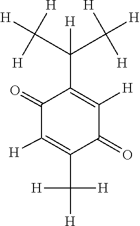 [SMILES: CC1=CC(=O)C(C(C)C)=CC1=O] | −6.3 | Pro98 His261 | Phe97 (Pi sigma) Phe97 Tyr324 | His210 Phe262 Val263 | Ile217 Pro292 |

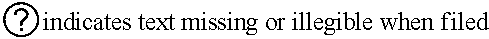

TABLE 2
Thermodynamic parameters for complexes of lead
compounds and positive controls with 3FMQ
(target) as computed by MM-GBSA method.

| | Delta Total | | |
|---|---|---|---|
| Compound | (kCal/mol) | SD | SEM |
| MetAPs-Carvacrol | −13.9848 | 1.7991 | 0.0804 |
| MetAPs-α-terpineol | −9.9831 | 2.4031 | 0.1074 |
| MetAPs-Thymoquinone | −1.3723 | 2.7601 | 0.1233 |
| MetAPs-Fumagillin | −28.2391 | 6.9002 | 0.3083 |
| MetAPs-Albendazole | −14.5590 | 2.5104 | 0.1122 |

TABLE 3
Thermodynamic parameters for complexes of lead compounds and positive
controls with MetAP2 (3FMQ) as computed by MM-PBSA method.

| | Van der | | Polar | | |
|---|---|---|---|---|---|
| | Waals | Electrostatic | salvation | SASA | Binding |
| | energy | energy | energy | energy | energy |
| Complexes | (kCal/mol) | (kCal/mol) | (kCal/mol) | (kCal/mol) | (kCal/mol) |
| MetAP2-Carvacrol | −20.29 ± 1.55 | −14.91 ± 2.89 | 30.43 ± 2.93 | −16.57 ± 0.72 | −21.34 ± 4.46 |
| MetAP2-α-terpineol | −21.42 ± 1.93 | −3.81 ± 6.80 | 18.14 ± 7.68 | −24.39 ± 1.00 | −12.64 ± 10.48 |
| MetAP2- | | | | | |
| Thymoquinone | −2.93 ± 4.46 | −0.79 ± 2.70 | 2.80 ± 4.68 | −2.20 ± 4.27 | −3.12 ± 8.20 |
| MetAP2-Fumagillin | −35.44 ± 3.33 | −41.70 ± 12.05 | 59.46 ± 8.50 | −32.36 ± 2.66 | −50.05 ± 15.35 |
| MetAP2-Albendazole | −19.68 ± 2.25 | −9.18 ± 3.08 | 18.92 ± 3.18 | −14.35 ± 1.51 | −24.30 ± 5.19 |

All the values are represented as mean ± SD.

### Example 2

The mechanism of EHP infection involves extrusion of polar tube (adhesive mechanism). In an in vitro study, inactivation of polar tube extrusion of EHP spores were tested with selected active ingredients namely thymoquinone, cinnamic aldehyde, eugenol, and carvacrol. The stock solution of bioactive molecules was prepared in dimethyl sulfoxide (DMSO) and further working solution prepared with 1× sterile phosphate buffer saline (1×PBS).

EHP spores were isolated and purified from HP of infected shrimps from high saline shrimp culture system (Batch 1) and low saline shrimp culture system (Batch 2). Purified spores were then incubated with the test molecules at a desired concentration for 2 hours at 37° C. Washed spores were then induced for polar tube extrusion with alkaline agent. The extrusion of the spore polar tubes was determined under a light microscope at 100× magnification by counting the number of extruded spores from a total of 100 spores and converting the number into % of extrusion.

In the results of Batch 1 spores, thymoquinone at 10 ppm and 100 ppm showed significantly reduced polar tube extrusion rate, 43.92% and 6.34% respectively, as depicted in FIG. 4. Similar results replicated in Batch 2 spore study and the effect of thymoquinone observed in dose-dependent in both Batch 1 and Batch 2 results. Compared to the 86.47% and 88.32% of extrusion of the 2% and 20% DMSO controls, only 40.31% and 0.31% were found post-incubation with 10 and 100 ppm thymoquinone, respectively, as depicted in FIGS. 1 and 2. The photomicrographs of polar tube extruded-EHP spores under microscopic examination of Batch 1 and Batch 2 study are represented in FIG. 3. The results clearly demonstrate that compositions containing thymoquinone would be a promising candidate for controlling EHP infection in shrimp by its ability to inhibit the polar tube extrusion. Other bioactive molecules such as cinnamic aldehyde, carvacrol and eugenol had a slight reduction on polar tube extrusion of EHP spores at higher concentration 100 ppm compared to DMSO control (p>0.05).

Statistical analysis. The One-way analysis of variance (ANOVA) and t-Test were performed to determine the significant differences at a 95% confidence interval of % polar tube extrusion between spores treated with bioactive molecules and the corresponding DMSO control. The analyses were carried out using STATGRAPHICS centurion.

### Example 3

An in vivo study (40 days) was conducted in shrimp with uninfected and infected controls and two treatment groups. The treatment groups included: Nigella sativa extract at 0.5% (Prototype 1), and a combination of cinnamic aldehyde and oregano oil (Prototype 2). In the experimental design, each group included 3 replicates, and each replicate included 15 shrimp. Water exchange (20-30%) was completed every two days. The aquarium tanks were maintained at recommended temperature (28-30° C.) and dissolved oxygen (>5 mg/L). The experimental diets were fed to shrimp 3 times a day at 3 to 3.5% of body weight for 40 days.

The shrimp were screened for EHP and other shrimp pathogens by PCR testing methods. Animals which were EHP positive but free from other infections were selected for infected and treatment groups. Same age shrimps negative for EHP, and other infection served as an uninfected control.

Data collection. Data was collected to consider: (1) Shrimp immune status. Total haemocytes count (THC) was measured by counting the cells in a drop of anticoagulant mixed hemolymph, placed under microscope. (2). Hepatopancreases health marker enzymes. Aspartate aminotransferase (AST) and Alkaline phosphatase (AP) were measured in shrimp hemolymph by spectrophotometric method. (3). Quantification of EHP. Shrimp hepatopancreas was dissected aseptically and the DNA was extracted and quantified the EHP infection by qPCR.

Results. The treatment groups showed significantly higher (p<0.05) total haemocyte count than controls indicating the enhanced health status of shrimp as represented in FIG. 4. Elevated levels of hepatic marker enzymes in hemolymph indicate the acute damage of HP. The marker enzymes, AST and AP were significantly (p<0.05) lower in treatments compared to infected control from day 20 as represented in FIGS. 5 and 6. EHP DNA quantification by qPCR showed a gradual increase of threshold cycle values in treatment groups from day 0 to 40 compared to infected control. Prototype-1 at 0.5% inclusion rate showed a significant increase in threshold cycle value with lower EHP DNA copy numbers (Table 4).

TABLE 4
EHP quantification in hepatopancreas - RT PCR

| | Day 1 | | Day 10 | | Day 20 | | Day 30 | | Day 40 | |
|---|---|---|---|---|---|---|---|---|---|---|
| | EHP | Copy | EHP | Copy | EHP | Copy | EHP | Copy | EHP | Copy |
| Groups | cycles | numbers | cycles | numbers | cycles | numbers | cycles | numbers | cycles | numbers |
| Infected control | 21.6 | 6048 | 21.8 | 2870 | 22.13 | 1811 | 23.2 | 1647 | 23.4 | 1615 |
| Prototype 1 | 21.6 | 6048 | 23.5 | 1600 | 25.9 | 1244 | 30.4 | 571 | 31.5 | 407 |
| (N. sativa extract 0.5%) | | | | | | | | | | |
| Prototype 2 | 21.6 | 6048 | 24 | 1526 | 25.2 | 1347 | 27.6 | 990 | 29.1 | 762 |
| (Aquavibra) | | | | | | | | | | |

### Example 4

An in vivo study (40 days) was conducted in shrimp with uninfected and infected controls and three treatment groups. The treatment groups included: Nigella sativa seed oil at 0.5% (Prototype 1), and a combination group (Prototype 2) at two different inclusion rates (0.5% and 1% by weight in the feed). The combination prototype was prepared containing N. sativa oil, oregano oil, clove oil and cinnamaldehyde. In the experimental design, each group 4 replicates, and each replicate 40 shrimp were maintained. Water exchange (20-30%) was completed every two days. The aquarium tanks were maintained at recommended temperature (28-30° C.) and dissolved oxygen (>5 mg/L). The experimental diets were fed to shrimp 3 times a day at 3-3.5% of body weight for 40 days. Post feeding trial, the animals were subjected to Vibrio parahaemolyticus challenge with LD 30 dosage.

For the experiment, shrimp were screened for EHP and other shrimp pathogens by PCR testing methods. Animals which were EHP positive but free from other infections were selected for infected and treatment groups. Same age shrimps negative for EHP, and other infection served as an uninfected control.

Data collection. Data was collected to consider: (1) Shrimp immune status. Total hemocyte count (THC), prophenoloxidase activity (proPO) and superoxide anion dismutase (SOD) activity were evaluated at 10 days intervals. THC was measured by counting the cells in a drop of anticoagulant mixed hemolymph, placed under microscope. ProPO activity was measured spectrophotometrically, measuring the formation of dopachrome from L-dihydroxyphenylalanine (L-dopa). SOD was evaluated by a spectrophotometric method based on epinephrine autoxidation. (2) Immune gene expression. The immune gene expression profiles of penaedin 3a, crustin, anti-lipopolysaccharide factor, toll-like receptor, and β-actin (housekeeping gene) was studied by qRT-PCR. The qRT-PCR was performed in triplicates. Relative quantification was assessed using Ct values. For each sample, the Ct value of the target genes was subtracted with the value of housekeeping gene (β-actin) to normalize the relative expression levels of the genes to arrive at the ΔCt values. The ΔCt values of test samples (infected control and treatment groups) was subtracted with the ΔCt values of caliber sample (uninfected control) to arrive the ΔΔCt values. (3) Hepatopancreases health marker enzymes. Alanine transaminase (ALT), Aspartate aminotransferase (AST) and Alkaline phosphatase (AP) were measured in shrimp hemolymph by the spectrophotometric method. (4) Quantification of EHP. Shrimp hepatopancreas was dissected aseptically and the DNA was extracted and quantified the EHP by qPCR. (5) Experimental immersion challenge with V. parahaemolyticus. Post feeding study, the shrimps were transferred to challenge tanks (100 L capacity and 60 L water) and similar salinity, aeration and respective feed were followed. In each tank, 30 shrimp were stocked and acclimatized for 40 hours. Field isolate of V. parahaemolyticus used for the immersion challenge study. After completion of the acclimatization period, shrimp were immersed at the LC30 dose (2.6×10³ CFU/mL) of V. parahaemolyticus suspension, while sterilized seawater replaced the bacterial suspension in the uninfected group. The mortality rate of shrimp was recorded every 6 h up to 5 days of post-immersion.

Results. The total haemocyte count showed a significant reduction as the disease progressed in the infected control compared to treatment groups from day 20. Treatment groups showed an initial reduction till day 20 and enhancing levels of THC in subsequent days as represented in FIG. 7. All the treatment groups showed significant (p<0.05) increase of THC from day 20 compared to infected control. All the treatment groups showed gradually decreased levels of ProPO and SOD activities from day 1 to 10 as represented in FIGS. 8 and 9. Infected control showed significantly higher levels of ProPO and SOD activities from day 10 to 40 compared to uninfected control and treatment groups.

Immune gene expression. The expression of penaeidin-3a was reduced in all the treatment groups compared to initial level. Whereas infected control showed higher expression of penaeidin-3a compared to initial level and significantly higher expression compared to other treatment groups at day 40 as represented in FIG. 10. The expression of crustin varied in different intervals. All the treatment groups showed lower expression of crustin levels compared to infected control at day 40. Among the treatment groups, the combination groups (Prototype 2) showed gradually decreased levels of crustin expression as depicted in FIG. 11. ALF and TLR expression were significantly (p<0.05) higher in Prototype 1 compared to infected control and other treatment groups from day 20 and 30 respectively. The ALF levels significantly lower in combination groups (Prototype 2) compared to other groups at day 40 as represented in FIG. 12. The TLR expression was significantly higher in Prototype 1 from day 30 compared to other groups as represented in FIG. 13.

Hepatopancreases health marker enzymes. The levels of aspartate aminotransferase, alanine aminotransferase and alkaline phosphatase in the haemolymph of infected and treatment groups were higher on day 1 compared to the uninfected group. On day 10 onwards, levels were significantly (p<0.05) lower in all the treatment groups compared to the infected control. Infected control had high levels of marker enzymes throughout the study duration. The AST and ALT levels were significantly lower in combination groups (Prototype 2) compared to infected control and other treatment group from day 10 as represented in FIGS. 14 and 15. The AP level was significantly (p<0.05) lower in combination groups compared to infected control and other treatment groups at day 40 as represented in FIG. 16.

Quantification of EHP. Treatment groups showed the gradual increase of C_{T} values compared to infected control. All the treatment groups showed lower copy numbers at day 40 compared to infected control (Table 5). Among the treatments, Prototype 2 (combination group at 5 g/kg of feed) showed a significantly lower copy numbers (35) compared to other groups.

TABLE 5
EHP quantification in hepatopancreas - RT PCR (Data represented as mean ± SD: N = 3 (p < 0.05))

| | Day 1 | | Day 10 | | Day 20 | | Day 30 | | Day 40 | |
|---|---|---|---|---|---|---|---|---|---|---|
| | EHP | Copy | EHP | Copy | EHP | Copy | EHP | Copy | EHP | Copy |
| Treatment | cycles | numbers | cycles | numbers | cycles | numbers | cycles | numbers | cycles | numbers |
| Infected | 23.3^{ab} ± 0.2 | 4706 | 22.5^{c} ± 0.4 | 5241 | 22.5^{c} ± 0.4 | 4982 | 22.2^{d} ± 0.7 | 5516 | 22.1^{d} ± 0.6 | 5605 |
| control | | | | | | | | | | |
| Prototype | 23.4^{ab} ± 0.2 | 4462 | 24.9^{b} ± 0.4 | 3434 | 24.9^{b} ± 0.4 | 3395 | 26.1^{c} ± 0.3 | 2065 | 26.5^{c} ± 0.3 | 1717 |
| 1 at 05% | | | | | | | | | | |
| Prototype | 23.63^{a} ± 0.2 | 4280 | 26.33^{a} ± 0.4 | 1886 | 26.33^{a} ± 0.4 | 743 | 28.93^{a} ± 0.5 | 154 | 30.06^{a} ± 0.5 | 35 |
| 2 at 0.5% | | | | | | | | | | |
| Prototype | 23.4^{ab} ± 0.3 | 4462 | 25.46^{ab} ± 0.4 | 2683 | 25.46^{ab} ± 0.4 | 1797 | 27.3b^{c} ± 0.4 | 1035 | 28.5^{b} ± 0.6 | 197 |
| 2 at 1% | | | | | | | | | | |

Experimental immersion challenge with V. parahaemolyticus. The infected control group showed significantly lower final survival rate compared to uninfected control and treatment groups as represented in FIG. 17. There was no significant difference between the treatment groups, however the combination groups (Prototype 2) showed improved survival compared to other treatment groups.

The researchers observed that the immune status improved in the treatment groups. The hemocytes are blood cells of invertebrates involved in key activities of innate immunity. Treatment groups showed improved hemocytes count following an initial decline representing increased disease resistance. The prophenoloxidase (proPO) cascade is an important innate immune response against microbial infections in invertebrates. The antioxidant enzyme superoxide dismutase (SOD) converts this microbiocidal metabolite into hydrogen peroxide that passes freely through membranes. The maintained initial levels of proPO and SOD activities in infected control and gradually reduced activities in the treatment groups indicates the recovery from the infection.

Antimicrobial peptides (AMPs) are part of an important first-line response in the host defense system. Cationic AMPs in penaeid shrimps composed of penaeidins, crustins, and anti-lipopolysaccharide factors are comprised of multiple classes or isoforms and possess antibacterial and antifungal activities against different strains of bacteria, fungi and enveloped virus. Penaeidins, which act mainly against Gram-positive bacteria and filamentous fungi, are composed of an N-terminal domain rich in proline residues and a C-terminal domain containing six cysteines that form three disulfide bridges. The expression of penaeidin-3a gradually reduced in all the treatment groups from initial level indicating the reduction of infection. Similarly, ALF and crustin expression reduced with combination groups demonstrated the lower bacterial and fungal infection in shrimps. Toll-like receptors (TLRs) are a class of proteins that play a crucial role in the innate immune system. They are single, membrane-spanning, non-catalytic receptors that recognize structurally conserved molecules derived from microbes. High relative expression of TLR in Nigella sativa alone and in combination groups demonstrated immunomodulatory effects of bioactive compounds present in the N. sativa seed/oil and its combination with essential oils.

Treatment groups showed significantly reduced hepatopancreas health marker enzymes such as AST, ALT and AP, which indicates the recovery of hepatopancreatic epithelial cells from damage caused by EHP infection. Previous study supports the elevated levels of AST and ALT in the hemolymph were related to necrosis and extensive damage in hepatopancreatic cells due to a severe infection of EHP and were observed in naturally and experimentally EHP-infected shrimp. Santhoshkumar, S., et al. (2017), Biochemical changes and tissue distribution of Enterocytozoon hepatopenaei (EHP) in naturally and experimentally EHP-infected white leg shrimp, Litopenaeus vannamei, J. Fish Dis. 40(4):529-539.

The lower production of HP health marker enzymes was in alignment with EHP DNA quantification results. qPCR results showed a gradual increase of threshold cycle values in treatment groups from day 0 to 40 compared to infected control. Combination group (Prototype 2) significantly (p<0.05) lowered the EHP DNA copy numbers, where it is understood that EHP-infected shrimp have a higher susceptibility to Vibrio related diseases. Luis, F. A., et al., (2017), Enterocytozoon hepatopenaei (EHP) is a risk factor for acute hepatopancreatic necrosis disease (AHPND) and septic hepatopancreatic necrosis (SHPN) in the Pacific white shrimp Penaeus vannamei, Aquaculture, 471, 37-42. Higher survival was observed in treatment groups against V. parahaemolyticus challenges indicating lower susceptibility to Vibrio infection.

### Example 5

Mature EHP spores are infective and are ubiquitous in shrimp culture systems from hatchery to grow out ponds. Consequently, healthy shrimps have a high probability of being exposed to mature spores and contracting the infection. The prototype was a blend of plant extracts which were tested in a co-habitation challenge model to evaluate the effect of supplementation in an environment of continuous and substantial exposure to mature EHP spores excreted by infected shrimp.

An in vivo trial (40 days) was conducted to investigate the effect of prototype (combination of Nigella sativa oil, Monarda fistulosa oil, Oregano vulgare oil, Syzygium aromaticum (clove) oil and cinnamic aldehyde) supplementation against Enterocytozoon hepatopenaei through co-habitation challenge in Litopenaeus vannamei. The trial design consisted of four groups, including two controls (uninfected control and infected control), and prototype was top-dressed on the feed at 0.3% and 0.5% dosage, with eight replicates each. In each replicate of infected control, prototype groups, EHP-infected shrimps were housed in cages, while healthy shrimps were stocked in outside the cage of the same tanks (FIG. 18A). Similar set up was followed for the uninfected group but had healthy shrimps both inside and outside the cage (FIG. 18B). The details of experimental groups are mentioned in Table 6. Measured the growth parameters and EHP infection rate at different intervals. All the tanks were maintained at the recommended temperature range of 28-30° C. and a dissolved oxygen level of over 5 mg/L. The experimental diets were fed to the shrimp three times a day at 3-3.5% of their body weight for a period of 40 days.

TABLE 6
Details of experimental groups.

| | EHP | | |
|---|---|---|---|
| Group | challenge | Cohabitation | Supplementation and dosage |
| Uninfected control | No | Inside cage: Healthy shrimp | Inside and outside cage shrimp: Basal diet |
| | | Outside cage: Healthy | |
| | | shrimp | |
| Infected control | Yes | Inside cage: EHP infected | Inside and outside cage shrimp: Basal diet |
| Prototype at 0.3% | Yes | shrimp | Inside cage shrimp: Basal diet |
| | | Outside cage: Healthy | Outside cage shrimp: Basal diet + |
| | | shrimp | Prototype at 0.3% |
| Prototype at 0.3% | Yes | | Inside cage shrimp: Basal diet |
| | | | Outside cage shrimp: Basal diet + |
| | | | Prototype at 0.5% |

Selection of experimental animal. The selection criteria for the infected animals were that they tested positive for EHP and negative for other shrimp pathogens. Animals that tested negative for all diseases were selected as healthy shrimp.

Data collection. (1). The growth performance parameters including weekly body weight gain, feed conversion ratio (FCR), specific growth rate (SGR) and survival rate of shrimps outside the cage were recorded. (2). Quantification of EHP—shrimp hepatopancreas was individually and aseptically dissected from each representative outside cage shrimp samples. One shrimp was taken from each replicate, and DNA was extracted for EHP quantification using qPCR.

Statistical analysis. The statistical differences between treatments were calculated by one-way ANOVA using STATGRAPHICS centurion software at significant level (p-value<0.05).

Results. The researchers observed that weekly weight gain was significantly higher (p<0.05) in the prototype supplemented groups compared to the infected control group at all the intervals (FIG. 19). By day 40, the final weight gain of the prototype at 0.5% group (11±1.96 g) was comparable to that of the uninfected control group (12.75±1.57 g). Furthermore, Prototype supplementation resulted in reduced FCR and increased SGR % compared to the infected control group, as depicted in FIG. 20. The survival rate was significantly improved (p<0.05) in the prototype supplemented groups (>80%) compared to the infected control group (70%) (FIG. 21).

The infected control group showed significantly higher Enterocytozoon hepatopenaei (EHP) DNA copy numbers (8650/μL) during the 5-day post-challenge period, and this higher infection level (with higher EHP DNA copies) was sustained in the infected control group. In contrast, the prototype supplemented groups showed negative PCR results at 5 days post-challenge and maintained significantly lower EHP copies throughout the entire 40-day challenge period compared to the infected control group. By day 40, the group supplemented with prototype at 0.5% exhibited a higher fold decrease in EHP copies (67-fold) compared to the group supplemented with prototype at 0.3% (12-fold) as represented in FIG. 22. This confirms the Prototype supplementation delays the EHP infection and significantly lower the infection rate under severe challenge conditions.

The results suggests that the prototype in this study, a composition containing Nigella sativa oil, Monarda fistulosa oil, Syzygium aromaticum oil, Oregano vulgare oil and cinnamic aldehyde, could be a functional ingredient in shrimp feed to increase disease resistance against EHP infection and serve as a protective tool against shrimp production loss caused by EHP infection.

### Example 6

The researchers investigated the effect of the prototype (combination of Nigella sativa oil, Monarda fistulosa oil, Syzygium aromaticum oil, Oregano vulgare oil and cinnamic aldehyde) supplementation against Enterocytozoon hepatopenaei in L. vannamei reared in earthen shrimp culture system. The initial EHP infection rate confirmed by qPCR. prototype was top-dressed onto commercial feed using a suitable binder and administered to infected shrimp at an inclusion rate of 0.5% and 1% in their diet for a duration of 30 days. The infection rate was measured at regular intervals. After the initial 30-day period, prototype supplementation was discontinued, and the shrimp were fed a control diet for 20 days to assess the reinfection rate. This was done by quantifying the EHP DNA copies in the shrimp hepatopancreas using qPCR. Also, the growth parameters including biomass yield, FCR, SGR and survival was recorded and compared with adjacent non-EHP infected shrimp culture system (control).

TABLE 7
Quantification of EHP infection by qPCR.

| | | | | | | | | | | | Observation period | |
|---|---|---|---|---|---|---|---|---|---|---|---|---|
| | Prototype supplementation period (DOC 49-81) | | | | | | | | | | (DOC 82-99) | |
| | Day 0 | | Day 7 | | Day 14 | | Day 21 | | Day 28 | | Day 46 | |
| | Ct | Copy | Ct | Copy | Ct | Copy | Ct | Copy | Ct | Copy | Ct | Copy |
| Groups | Value | numbers | Value | numbers | Value | numbers | Value | numbers | Value | numbers | Value | numbers |
| Control | ND | ND | ND | ND | ND | ND | ND | ND | ND | ND | ND | ND |
| Prototype at 1% | 20.5 | 7312 | 21.1 | 6587 | 25.7 | 2421 | ND | ND | ND | ND | ND | ND |
| Prototype at 0.5% | 21.8 | 5731 | 27.3 | 1035 | ND | ND | ND | ND | ND | ND | ND | ND |

The inventors observed a significant decrease of EHP infection in prototype supplemented ponds as mentioned in Table 7. The growth, survival and feed efficiency were improved with prototype supplementation compared to control pond as depicted in FIGS. 23 and 24. Further, the size variations reduced in prototype supplemented shrimps as represented in FIG. 25.

As described throughout this disclosure, the inventors have surprisingly found that N. sativa seed extract/oil alone or in combination with one or more essential oils including but not limited to oregano oil, monarda oil, clove oil and cinnamic aldehyde resulted in a synergistic effect in controlling and inhibiting the spread of EHP infection and its associated diseases.

In at least one embodiment, the inventors have surprisingly discovered a method for controlling the spread of EHP infection and associated diseases in shrimp comprising orally administering to the shrimp a composition containing thymoquinone in an amount effective to reduce the EHP infection rate. In certain embodiments, the thymoquinone is present in an amount of at least 100 ppm, for instance 200 ppm, 300 ppm, 400 ppm, or 500 ppm. In certain embodiments, the thymoquinone is derived from Monarda didyma, Monarda fistulosa, or Nigella sativa.

In at least one embodiment, the compositions of the present invention further comprise one or more essential oil selected from the group consisting of monarda oil, oregano oil, clove oil and cinnamic aldehyde. By way of non-limiting example, in at least one embodiment, the composition of the present invention comprises thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

In at least one embodiment, the composition of the present invention is administered by adding the composition to the animal diet, for instance the composition is added to the shrimp's diet as an animal feed additive. For instance, the composition is added to the feed in an amount ranging from about at least 0.1 percent, and in certain embodiments from about 0.1 to about 2 percent by weight of the feed.

In alternative embodiments, the composition is added to the water or pond, wherein the composition is administered in an amount effective to inactivate the EHP spores present in the shrimp culture water and shrimp pond soil sediments.

The inventors have also discovered a method for inactivation of EHP spores comprising orally administering to the shrimp a composition containing thymoquinone, for instance a composition comprising Nigella sativa seed extract/oil and one or more essential oils/extracts in an amount effective to inactivate the EHP spores and reduce the EHP infection rate. In certain embodiments, the composition of the present invention further comprises the one or more essential oil, including but not limited to monarda oil, oregano oil, clove oil and cinnamic aldehyde/cinnamon oil. In at least one embodiment, the composition of the present invention comprises thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

Another embodiment of the present invention relates to a feed additive composition comprising thymoquinone in an amount effective to inactivate the EHP spores and reduce the EHP infection rate. In certain embodiments, the thymoquinone is derived from Monarda didyma, Monarda fistulosa, or Nigella sativa. In certain embodiments, the feed additive contains Nigella sativa extract or oil. In at least one embodiment, the feed additive further comprises one or more essential oil including but not limited to monarda oil, oregano oil, clove oil and cinnamic aldehyde/cinnamon oil. For instance, in at least one embodiment, the composition of the present invention comprises thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

In certain embodiments, the feed additive is added to the shrimp diet as an animal feed additive, for instance in an amount of at least about 0.1 percent by weight of the feed, and in at least one embodiment in an amount ranging from about 0.1 to about 2 percent by weight of the feed. In alternative embodiments, the feed additive is added to the water or pond, wherein the composition is administered in an amount effective to inactivate the EHP spores present in the shrimp culture water and shrimp pond soil sediments.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

It should be further appreciated that minor dosage and formulation modifications of the composition and the ranges expressed herein may be made and still come within the scope and spirit of the present invention.

It is also to be understood that the formulations and processes illustrated in the attached drawings, and described in the following specification are simply exemplary embodiments of the inventive concepts defined in the appended claims. Hence, specific dimensions and other physical characteristics relating to the embodiments disclosed herein are not to be considered as limiting, unless the claims expressly state otherwise. Where a range of values is provided, it is understood that each intervening value, to the tenth of the unit of the lower limit unless the context clearly dictates otherwise, between the upper and lower limit of that range, and any other stated or intervening value in that stated range, is encompassed within the scope of the present disclosure. The upper and lower limits of these smaller ranges may independently be included in the smaller ranges, and are also encompassed within the scope of the present disclosure, subject to any specifically excluded limit in the stated range. Where the stated range includes one or both of the limits, ranges excluding either or both of those included limits are also included in the scope of the present disclosure. All ranges and parameters, including but not limited to percentages, parts, and ratios, disclosed herein are understood to encompass any and all sub-ranges assumed and subsumed therein, and every number between the endpoints. For example, a stated range of “1 to 10” should be considered to include any and all sub-ranges beginning with a minimum value of 1 or more and ending with a maximum value of 10 or less (e.g., 1 to 6.1, or 2.3 to 9.4), and to each integer (1, 2, 3, 4, 5, 6, 7, 8, 9, 10) contained within the range. In this specification and the appended claims, the singular forms “a,” “an” and “the” include plural reference unless the context clearly dictates otherwise. All combinations of method steps or process steps as used herein can be performed in any order, unless otherwise specified or clearly implied to the contrary by the context in which the referenced combination is made

To the extent that the terms “includes” or “including” or “have” or “having” are used in the specification or the claims, it is intended to be inclusive in a manner similar to the term “comprising” as that term is interpreted when employed as a transitional word in a claim. Furthermore, to the extent that the term “or” is employed (e.g., A or B) it is intended to mean “A” or “B” or both “A” and “B”. When the Applicant intends to indicate “only A or B but not both” then the term “only A or B but not both” or similar structure will be employed. Thus, use of the term “or” herein is the inclusive, and not the exclusive use. Also, to the extent that the terms “in” or “into” are used in the specification or the claims, it is intended to additionally mean “on” or “onto.” In this specification and the appended claims, the singular forms “a,” “an” and “the” include plural reference unless the context clearly dictates otherwise.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A method for controlling the spread of EHP infection and associated diseases in shrimp comprising administering to the shrimp a composition containing thymoquinone in an amount effective to reduce the EHP infection rate.

2. The method of claim 1, wherein the thymoquinone is present in an amount of at least 100 ppm.

3. The method of claim 1, wherein the thymoquinone is derived from Monarda didyma, Monarda fistulosa, or Nigella sativa.

4. The method of claim 1 further comprising one or more essential oil selected from the group consisting of monarda oil, oregano oil, clove oil and cinnamic aldehyde.

5. The method of claim 1, wherein the composition comprises thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

6. The method of claim 1, wherein the composition is added to the shrimp's diet as an animal feed additive.

7. The method of claim 1, wherein the composition is added to the water or pond, wherein the composition is administered in an amount effective to inactivate the EHP spores present in the shrimp culture water and shrimp pond soil sediments.

8. The method of claim 1, wherein the composition is added to the feed in an amount ranging from about 0.1 to about 2 percent by weight of the feed.

9. The method of claim 1, wherein the composition is added to the feed in an amount ranging of at least about 0.1 percent by weight of the feed.

10. A method for inactivation of EHP spores comprising administering to the shrimp a composition containing Nigella sativa seed extract/oil and one or more essential oils/extracts in an amount effective to inactivate the EHP spores and reduce the EHP infection rate.

11. The method of claim 10, wherein the one or more essential oil is selected from the group consisting of monarda oil, oregano oil, clove oil and cinnamic aldehyde/cinnamon oil.

12. The method of claim 10, wherein the composition comprises thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

13. The method of claim 10, wherein the thymoquinone is derived from Monarda didyma, Monarda fistulosa, or Nigella sativa.

14. The method of claim 10, wherein the composition is added to the shrimp's diet as an animal feed additive.

15. The method of claim 10, wherein the composition is added to the water or pond, wherein the composition is administered in an amount effective to inactivate the EHP spores present in the shrimp culture water and shrimp pond soil sediments.

16. The method of claim 10, wherein the composition is added to the feed in an amount ranging from about 0.1 to about 2 percent by weight of the feed.

17. The method of claim 10, wherein the composition is added to the feed in an amount ranging of at least about 0.1 percent by weight of the feed.

18. A feed additive composition comprising thymoquinone in an amount effective to inactivate the EHP spores and reduce the EHP infection rate.

19. The feed additive of claim 18, wherein the thymoquinone is derived from Monarda didyma, Monarda fistulosa, or Nigella sativa.

20. The feed additive of claim 18, wherein the additive contains Nigella sativa extract or oil.

21. The feed additive of claim 18, further comprising one or more essential oil is selected from the group consisting of monarda oil, oregano oil, clove oil and cinnamic aldehyde/cinnamon oil.

22. The feed additive of claim 18, wherein the composition contains thymoquinone in an amount ranging from about 0.01 to about 100% by weight, carvacrol in an amount ranging from about 0 to about 70% by weight, α-terpineol in an amount ranging from about 0 to about 70% by weight, cinnamic aldehyde in an amount ranging from about 0 to about 30% by weight, and eugenol in an amount ranging from 0 to about 30% by weight.

23. The feed additive of claim 18, wherein the composition is added to the shrimp diet as an animal feed additive.

24. The feed additive of claim 18, wherein the composition is added to the water or pond, wherein the composition is administered in an amount effective to inactivate the EHP spores present in the shrimp culture water and shrimp pond soil sediments.

25. The feed additive of claim 18, wherein the composition is added to the feed in an amount ranging of at least about 0.1 percent by weight of the feed.
